(19) United States
(12) Patent Application Publication
(10) Pub. No.: US 2022/0387623 A1
(43) Pub. Date: Dec. 8, 2022

(54) GROWTH FACTOR RESTORATION

(71) Applicant: Remedium Bio, Inc., Needham, MA (US)

(72) Inventor: Frank Luppino, Needham, MA (US)

(21) Appl. No.: 17/750,224

(22) Filed: May 20, 2022

Related U.S. Application Data
(63) Continuation of application No. PCT/US2020/061486, filed on Nov. 20, 2020.
(60) Provisional application No. 62/938,879, filed on Nov. 21, 2019.

(51) Int. Cl. A61K 48/00; C07K 14/50; C12N 15/86; A61P 19/04

(52) U.S. Cl. CPC A61K 48/005; C07K 14/50; C12N 15/86; A61P 19/04; C12N 2750/14143; C12N 2750/14171; C12N 2740/15043; C12N 2740/15071; A61K 38/00

(57) ABSTRACT

The present invention provides compositions and methods for treating cartilage disorders using expression vectors including a nucleic acid encoding a suicide gene, and a fibroblast growth factor 18 (FGF-18) polypeptide or a functional fragment thereof. The present invention relates to expression vectors, and methods of use thereof for promoting the proliferation of cells responsible for the production and maintenance of tissues, such as chondrocytes, cardiomyocytes, and synoviocytes.

### RELATED APPLICATIONS

The present application claims benefit of the filing date of U.S. Provisional Application No. 62/938,879, filed Nov. 21, 2019, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 19, 2020 is named “51518-003WO2_Sequence_Listing_11_19_20_ST25” and is 13,487 bytes in size.

### FIELD OF THE INVENTION

The invention relates to the field of therapeutic treatment of tissue disorders and specifically cartilage disorders in patients, such as human patients.

### BACKGROUND OF THE INVENTION

Growth factors (e.g., Fibroblast Growth Factor 18) are proteins that regulate cell proliferation, migration, survival, differentiation, tissue deposition, turnover, and maintenance, among many other biological functions. In general, growth factor levels in tissues decline as a function of age. This decline is due in part to reduced gene expression mediated by one of many mechanisms of genetic silencing, general reduction in the cell density (which is hypothesized as a positive feedback loop with growth factor level decline), decreased efficiency and effectiveness in translation, and an increased proportion of senescent cells. Cellular density in tissues is correlated with tissue composition and physicochemical properties. At least some of the decline in growth factor concentration has been associated with disease, tissue atrophy, and tissue degeneration. While growth factor supplementation, substitution, or the replacement of defective or absent growth factors are widely used approaches to restore cartilage degraded by osteoarthritis, there continues to be a need for improved therapeutic approaches.

### SUMMARY OF THE INVENTION

The present invention relates to expression vectors, and methods of use thereof for promoting the proliferation of cells responsible for the production and maintenance of tissues, such as chondrocytes, cardiomyocytes, and synoviocytes. The compositions and methods provided herein can be useful in the treatment or prevention of cartilage disorders associated with disease, tissue atrophy, and tissue degeneration. In some embodiments, the cartilage disorder is osteoarthritis.

In a first aspect, the invention features a recombinant expression vector including a nucleic acid encoding a suicide gene, and a Fibroblast Growth Factor 18 (FGF-18) polypeptide or a functional fragment thereof.

In some embodiments of the first aspect, the expression vector is a viral vector.

In some embodiments of the first aspect, the expression vector includes a non-viral particle and a nucleic acid encoding (1) a suicide gene, and (2) a FGF-18 polypeptide or a functional fragment thereof.

In some embodiments of the first aspect, the expression vector includes a hybrid viral and non-viral particle and a nucleic acid encoding (1) a suicide gene, and (2) a FGF-18 polypeptide or a functional fragment thereof.

In some embodiments, the viral vector is selected from an adeno-associated virus (AAV), an adenovirus, a parvovirus, a coronavirus, a rhabdovirus, a paramyxovirus, a picornavirus, an alphavirus, a herpes virus, a poxvirus, a lentivirus, and a Retroviridae family virus.

In some embodiments of any of the foregoing aspects, the viral vector is an AAV.

In some embodiments of any of the foregoing aspects, the AAV further includes a capsid.

In some embodiments of any of the foregoing aspects, the capsid includes a naturally occurring capsid or an engineered capsid (e.g., a capsid that contains a modified sequence).

In some embodiments of any of the foregoing aspects, the engineered capsid is conjugated to a ligand (e.g., for cell type specificity or modulation of immunogenicity).

In some embodiments of any of the foregoing aspects, the naturally occurring capsid is an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/6, DJ/9, 7m8, PHP.B, PHP.eB, or PHP.S capsid.

In some embodiments of any of the foregoing aspects, the AAV is an AAV2 or an AAV5.

In some embodiments of any of the foregoing aspects, the suicide gene includes a Herpes Simplex Virus-1 Thymidine Kinase (HSV-TK) gene, a Caspase 9 (Casp9) gene, a cytosine deaminase gene, a RQR85 polypeptide, or a truncated human EGFR polypeptide.

In some embodiments of any of the foregoing aspects, the suicide gene is a HSV-TK gene.

In some embodiments of any of the foregoing aspects, the suicide gene is a Casp9 gene.

In some embodiments of any of the foregoing aspects, the suicide gene encodes an amino acid sequence that has at least 85% sequence identity (e.g., at least 86%, at least 87%, at least 88%, at least 89%, at least 90%, at least 91%, at least 92%. at least 93%, at least 94. at least 95%, at least 96%, at least 97%, at least 98%, or at least 99% identity) to the amino acid sequence of SEQ ID NO: 1.

In some embodiments of any of the foregoing aspects, the suicide gene refers to a gene that causes a cell that expresses the expression vector to undergo programed cell death.

In some embodiments of any of the foregoing aspects, the suicide gene refers to a gene that causes a cell that expresses the expression vector to terminate the transcription or translation of the expression vector.

In some embodiments of any of the foregoing aspects, the suicide gene refers to a gene that causes a reduction in the expression level of the expression vector in a cell that expresses the expression vector.

In some embodiments of any of the foregoing aspects, the suicide gene is an inducible suicide gene.

In some embodiments of any of the foregoing aspects, the inducible suicide gene is a rapamycin-activated Casp9.

In some embodiments of any of the foregoing aspects, the suicide gene encodes an amino acid sequence that has at least 85% sequence identity (e.g., at least 86%, at least 87%, at least 88%, at least 89%, at least 90%, at least 91%, at least 92%, at least 93%, at least 94, at least 95%, at least 96%, at least 97%, at least 98%, or at least 99% identity) to the amino acid sequence of SEQ ID NO: 2.

In some embodiments of any of the foregoing aspects, the FGF-18 polypeptide encodes an amino acid sequence that has at least 85% sequence identity (e.g., at least 86%, at least 87%, at least 88%, at least 89%, at least 90%, at least 91%, at least 92%, at least 93%, at least 94, at least 95%, at least 96%, at least 97%, at least 98%, or at least 99% identity) to the amino acid sequence of SEQ ID NO. 3.

In some embodiments of any of the foregoing aspects, the expression vector further includes a transcriptional regulator operably linked to a nucleic acid encoding FGF-18 or a functional fragment thereof.

In some embodiments of any of the foregoing aspects, the expression vector further includes a non-viral particle.

In some embodiments of any of the foregoing aspects, the transcriptional regulator includes any one or more of a TATA box, a B recognition element recognition element, a transcription factor II B recognition element, and a downstream promoter element.

In some embodiments of any of the foregoing aspects, the non-viral particle includes any one or more of a lipid envelope, a phospholipid bilayer, a liposome, a noisome, a fullerene, a protein-based nanoparticle, a nanocrystal, a dendrimer, an organic/inorganic colloid, a pegylated liposome, a polymeric micelle, a reverse micelle, a mixed micelle, a sensitive micelle, a multicomponent micelle, or a poly- or uni-molecular dendritic micelle.

In another aspect, the invention features a pharmaceutical composition including the expression vector of any of the foregoing aspects.

In some embodiments of any of the foregoing aspects, the pharmaceutical composition further includes a pharmaceutically acceptable carrier, diluent, or excipient.

In another aspect, the invention provides a method of treating a cartilage disorder, the method including administering to the subject the pharmaceutical composition of any one of the foregoing aspects.

In another aspect, the invention provides a pharmaceutical composition of any one of the foregoing aspects for use in treating a cartilage disorder.

In another aspect, the invention provides the use of the pharmaceutical composition of any of the foregoing aspects for treating a cartilage disorder.

In another aspect, the invention provides use the pharmaceutical composition of any of the foregoing aspects in the manufacture of a medicament for treating a cartilage disorder. In some embodiments of any of the foregoing aspects, the cartilage disorder is osteoarthritis.

In some embodiments of any of the foregoing aspects, the pharmaceutical composition is administered intravenously, intra-articularly, sub-chondrally, intra-synovially, or intra-chondrally.

In some embodiments of any of the foregoing aspects, the pharmaceutical composition is administered to the synovial joint.

In another aspect, the invention provides a method of promoting proliferation in a chondrocyte cell or a chondrocyte precursor cell (e.g., by increasing survival of a chondrocyte cell or a chondrocyte precursor cells or by increasing differentiation of a chondrocyte precursor cell), the method including contacting a cell with the expression vector or the pharmaceutical composition of any of the foregoing aspects.

In another aspect, the invention provides an expression vector or a pharmaceutical composition of any of the foregoing aspects for use in promoting proliferation of a chondrocyte cell or a chondrocyte precursor cell (e.g., by increasing survival of a chondrocyte cell or a chondrocyte precursor cells or by increasing differentiation of a chondrocyte precursor cell).

In another aspect, the invention provides use of the expression vector or the pharmaceutical composition of any of the foregoing aspects for promoting proliferation of a chondrocyte cell (e.g., by increasing survival of a chondrocyte cell or a chondrocyte precursor cells or by increasing differentiation of a chondrocyte precursor cell). In another aspect, the invention provides use of the expression vector or the pharmaceutical composition of any of the foregoing aspects in the manufacture of a medicament for promoting proliferation of a chondrocyte cell or a chondrocyte precursor cell (e.g., by increasing survival of a chondrocyte cell or a chondrocyte precursor cells or by increasing differentiation of a chondrocyte precursor cell).

In another aspect, the invention provides a method of promoting the secretion of extracellular matrix to replace cartilage (e.g., by promoting proliferation of a chondrocyte cell or a chondrocyte precursor cell, by increasing survival of a chondrocyte cell or chondrocyte precursor cell, or by increasing differentiation of a chondrocyte precursor cells) the method including contacting a cell with the expression vector or the pharmaceutical composition of any of the foregoing aspects.

In another aspect, the invention provides an expression vector or a pharmaceutical composition of any of the foregoing aspects for use in promoting the secretion of extracellular matrix to replace cartilage (e.g., by promoting proliferation of a chondrocyte cell or a chondrocyte precursor cell, by increasing survival of a chondrocyte cell or chondrocyte precursor cell, or by increasing differentiation of a chondrocyte precursor cells).

In another aspect, the invention provides the use of the expression vector or the pharmaceutical composition of any of the foregoing aspects for promoting the secretion of extracellular matrix to replace cartilage (e.g., by promoting proliferation of a chondrocyte cell or a chondrocyte precursor cell, by increasing survival of a chondrocyte cell or chondrocyte precursor cell, or by increasing differentiation of a chondrocyte precursor cells).

In another aspect, the invention provides use of the expression vector or the pharmaceutical composition of any of the foregoing aspects in the manufacture of a medicament for promoting the secretion of extracellular matrix to replace cartilage.

In some embodiments of any of the foregoing aspects, cells are transfected or transduced ex vivo to express the expression vector, In another aspect, the invention features a kit including the expression vector or the pharmaceutical composition of any one of the foregoing aspects, and a package insert, wherein the package insert instructs a user of the kit to perform the method of any one of the foregoing aspects. In another aspect, the invention features a kit of the foregoing aspects, wherein the kit further includes one or more of a carton, a tamper evident seal, a needle, a syringe, or a prefixed syringe.

### BRIEF DESCRIPTION OF THE DRAWINGS

FIG. 1 is a schematic drawing of an exemplary adeno-associated virus 2 (AAV2) viral vector for the continual expression of an intrinsically safe Fibroblast Growth Factor 18 (FGF18) polypeptide, as mediated by the herpes simplex virus-1 thymidine kinase (HSV-TK) suicide gene. Dark shaded boxes represent the nucleic acid sequences encoding a cytomegalovirus (CMV)-enhancer (CMVenh) operatively linked to a human FGF18 (hFGF18) polypeptide, a woodchuck hepatitis virus post-transcriptional regulatory element mutant 6 (WPREmut6; a DNA element known to substantially increase expression levels but without promoter activity), a bovine growth hormone polyadenylation signal (bGHpA), a HSV-TK suicide gene, a mutated SV40 early polyadenylation signal (SV40pA), and flanking AAV2 inverted terminal repeat sequences (ITR). White boxes represent the CMV and phosphoglycerate kinase (PGK) promoters (CMVpr and PGKpr), respectively. The bold line joining the white and dark shaded boxes represents a minute virus of mice (MVM) intron for the optimized functionality of the CMV promoter.

FIG. 2 is a schematic drawing of an exemplary AAV2 viral vector for the continual expression of an intrinsically safe FGF-18 polypeptide, as mediated by the rapamycin-activated caspase 9-based (rapaCas9) suicide gene. Dark shaded boxes represent the nucleic acid sequences encoding a CMVenh operatively linked to a hFGF18 polypeptide, a WPREmut6, a bGHpA, a rapaCas9 suicide gene, a SV40pA, and flanking AAV2 ITR. White boxes represent CMVpr and PGKpr, respectively. The bold line joining the white and dark shaded boxes represents a MVM intron.

### DEFINITIONS

As used herein, “activity” refers to form(s) of a polypeptide which retain a biological activity of the native or naturally occurring polypeptide, wherein “biological” activity refers to a biological function (e.g., growth factor function) caused by a native or naturally-occurring polypeptide.

As used herein, “administration” refers to providing or giving a subject a therapeutic agent (e.g., an expression vector) by any effective route. Exemplary routes of administration are described herein and below (e.g., intravenous, intra-articular, sub-chondral, intra-synovial, or intrachondral injection).

As used herein, the term “allelic variant” refers to one of several possible naturally occurring alternate forms of a gene occupying a given locus on a chromosome of an organism or a population of organisms.

As used herein, a “cardiomyocyte” refers a muscle cell (myocyte) that makes up the cardiac muscle. As used herein, a “cartilage disorder” refers to a disorder presenting in patients as degeneration of cartilage; narrowing of the joint space; subchondral bone thickening; formation of osteophytes or bone spurs; inflammation in the joint accompanied by swelling and pain; reduction in the density of one or more cartilage tissue components, joint tissue components including cells, proteins, proteoglycans and polysaccharides; and other symptoms. In addition to mechanical damage, cartilage disorders are also associated with an increase in the levels of pro-inflammatory cytokines, such as tumor necrosis factor alpha, interleukin 6 (IL-6), and interleukin 1 (IL-1) beta. These cytokines can diffuse into cartilage and cause upregulation of protease activity, leading to degradation of multiple macromolecules of cartilage extracellular matrix by the matrix metalloproteinases, aggrecanases, hyaluronidases, and other tissue digesting enzymes.

As used herein, the term “cell type” refers to a group of cells sharing a phenotype that is statistically separable based on gene expression data. For example, cells of a common cell type (e.g., chondrocytes or synoviocytes) may share similar structural and/or functional characteristics, such as similar gene activation patterns and antigen presentation profiles. Cells of a common cell type may include those that are isolated from a common tissue (e.g. joint tissue) and/or those that are isolated from a common organ, tissue system, blood vessel, or other structure and/or region in an organism.

As used herein, the term “chondrocyte” refers to the cellular population that compose the primary cells found in healthy cartilage. Functionally, chondrocytes produce and maintain the cartilaginous matrix, which consists mainly of collagen and proteoglycans.

As used herein, the term “closed-end DNA” refers to linear duplex DNA with covalently closed ends.

As used herein, “codon optimization” refers a process of modifying a nucleic acid sequence in accordance with the principle that the frequency of occurrence of synonymous codons (e.g., codons that code for the same amino acid) in coding DNA is biased in different species, Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,688,112, each of which is incorporated herein by reference in its entirety. The sequence surrounding the translational start site can be converted to a consensus Kozak sequence according to known methods. See, e.g., Kozak et al, Nucleic Acids Res.15:8125-8148 (1989), incorporated herein by reference in its entirety. Multiple stop codons can be incorporated.

As used herein, a “combination therapy” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “doggybone DNA” refers to closed linear double-stranded DNA that is created through the enzymatic digestion of concatemeric DNA.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: an increase in the quantity or concentration of mRNA encoding a corresponding protein (as assessed, e.g., using RNA detection procedures known in the art, such as quantitative polymerase chain reaction and RNA sequencing techniques), and/or an increase in the quantity or concentration of a corresponding protein (as assessed, e.g., using protein detection methods described known in the art, such as Western blotting).

As used herein, the term “expression vector” refers to a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic ar eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of the expression vectors as described herein include plasmids that contain regulatory sequences, such as promoter and enhancer regions, which direct gene transcription. Other useful vectors for expression of expression vectors contain polynucleotide sequences that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

As used herein, the terms “Fibroblast Growth Factor 18,” “FGF-18,” and “FGF18” refer to a protein maintaining at least one biological activity of the human FGF-18 protein. As used herein. FGF-18 refers to FGF-18 in its native form, in its mature form, in a recombinant form, as well as in any other form of the naturally occurring FGF-18 variants (e.g., splice variants, allelic variants, or variants arising from post-translational modifications or secondary structures). As used herein, FGF-18 refers also to an FGF-18 protein including a fusion protein, wherein the hybrid protein maintains at least one biological activity of the human FGF-18 protein. As used herein, FGF-18 refers also to a modified FGF-18 protein, wherein the modification, for example, increases resistance to degradation or modulates its affinity for binding to one or more of its receptors. Biological activities of the human FGF-18 protein notably include the increase in chondrocyte or osteoblast proliferation (see WO98/16844) or cartilage formation (see WO2008/023083). Native human FGF-18 is a protein expressed by chondrocytes of articular cartilage. Human FGF-18 was first designated zFGF-5 and is fully described in WO98/16644. Human FGF-18 has NCBI Gene ID NO 8817. An exemplary wild-type human FGF-18 nucleic acid sequence is provided in NCBI RefSeq Acc. No. NM_003862.3 (SEQ ID NO: 3), and an exemplary wild-type FGF-18 amino acid sequence is provided in NCBI RefSeq Acc. No. NP_003853.1 (SEQ ID NO: 4).

As used herein, the term “functional fragment thereof” refers to an FGF-18 polypeptide that may be in its native form, in its mature form, in a recombinant form, in a form shorter than the full naturally occurring FGF-18 variants, Or may be in any other form of the naturally occurring FGF-18 variants (e.g., splice variants or allelic variants) and maintains at least one biological activity of the human FGF-18 protein.

As used herein, the term “IRES” refers to an internal ribosome entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

By “level” is meant a level of a protein or nucleic acid, as compared to a reference. The reference can be any useful reference, as defined herein. By a “decreased level” or an “increased level” of a protein or nucleic acid is meant a decrease or increase in protein or nucleic acid level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%. or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or nucleic acid in a sample.

As used herein, the term “mature form” refers to an FGF-18 polypeptide lacking a leader sequence and may also include other modifications of a polypeptide such as proteolytic processing of the amino terminus (with or without a leader sequence) and/or the carboxyl terminus, cleavage of a smaller polypeptide from a larger precursor. N-linked and/or O-linked glycosylation, and other post-translational modifications understood by those with skill in the art.

As used herein, the term “minicircle” refers to an episomal DNA vectors that is produced as circular expression cassette devoid of any bacterial plasmid DNA backbone.

As used herein, the term “ministring DNA” refers to a linear covalently closed DNA vector that includes only the gene of interest and necessary eukaryotic expression elements. It is devoid of immunogenic bacterial sequences.

As used herein, the term “nanoplasmid™” refers to a DNA vector with a backbone of less than 500 bp, no antibiotic markers, and a R6K origin of replication. It is available from Nature Technology Corporation.

As used herein, the terms “native” or “naturally occurring.” when used in connection with biological materials such as nucleic acid molecules, polypeptides, host cells, and the like, refers to that which are found in nature and not manipulated by a human being.

As used herein, “osteoarthritis” refers to a type of cartilage disorder that occurs when flexible tissue at the ends of bones wears down presenting as joint pain and stiffness, joint swelling, decreased range of motion, weakness or numbness of the arms and legs, and other symptoms.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a composition containing a nucleic acid described herein, formulated with a pharmaceutically acceptable excipient, and manufactured or sold with the approval of a governmental regulatory agency as part of a therapeutic regimen for the treatment of disease in a subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “programmed cell death” refers to the death of a cell as a result of events inside of a cell, such as apoptosis or autophagy.

As used herein, the term “promoter” refers to a recognition site on DNA that is bound by an RNA polymerase. The polymerase drives transcription of the transgene. Exemplary promoters suitable for use with the compositions and methods described herein. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, the term “splice variant” refers to a nucleic acid molecule, usually RNA, which is generated by alternative processing of intron sequences in an RNA transcript.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition. In some embodiments, the subject is a human.

As used herein, the term “suicide gene” refers to a gene that causes a cell to undergo programmed cell death, a gene that terminates the transcription or translation of the expression vector, itself, a gene that reduces the expression level of the expression vector through RNA interference, or a gene that encodes surface proteins (e.g., such as RQR85 and the truncated human Epidermal Growth Factor receptor polypeptide).

As used herein, the term “synoviocyte” refers to a fibroblast-like synoviocyte, which is a specialized cell type located inside joints in the synovium that produce synovial fluid glycoproteins essential for joint lubrication.

As used herein, the term “transcriptional regulator” refers to an inducible element that can regulate expression of a protein, e.g., FGF-18, in response to a stimulus (e.g., inflammation, cytokines, low levels of extracellular growth factors, infection, hypoxia, low or high levels of iron, low or high levels of ferritin, low or high levels of ampicillin, low or high levels of isopropyl β-d-1-thiogalactopyranoside, doxycycline, nitric oxide, nitric oxide synthases (NOS); paracrine, endocrine, or autocrine signaling; or external stimuli that cause tissue degeneration or infection that causes structural changes in the proximity of the cell carrying the expression vector).

As used herein, the terms “transduction” and “transduce” refer to a method of introducing an expression vector, e.g., a viral vector construct or a part thereof, into a cell and subsequent expression of a transgene encoded by the expression vector construct or part thereof in the cell.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, diethylaminoethyl (DEAE)-dextran transfection, NUCLEOFECTION™, squeeze-poration, sonoporation, optical transfection, MAGNETOFECTION™, impalefection, gold particle bombardment, and the like.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the term “U-rich motif” refers to a strand of consecutive nucleic acids in which at least 80% of the consecutive nucleic acids are uracil (U).

Advantages

The expression vectors and methods of use thereof exemplified herein provide several advantages over conventional therapies, which canonically include repeat intra-articular injections of the FGF-18 polypeptide for the promotion of chondrocyte and respective cartilage proliferation. These treatment regimens must be repeated continually to maintain cartilage gains, as rapid cartilage loss resumes upon discontinuation of injections. First, these compositions and methods permit continuing therapeutic expression of FGF-18 within the joint without the necessity of multiple treatments. Second, the expression vectors described herein encode a suicide gene which serves as a ‘safety switch’ mechanism to prevent overproduction or to allow conditional reduction of the level of FGF-18 expression. Therefore, the expression vectors and methods of use thereof exemplified herein overcome the limitations of traditional FGF-18 supplementation, by transducing cells of the synovial joint (e.g., synoviocytes and/or chondrocytes) with the genetic sequence of an FGF-18 polypeptide that is intrinsically-safe, as mediated by the inducible suicide gene, and enables the continual therapeutic regenerative properties mediated by FGF-18 expression.

### DETAILED DESCRIPTION

The compositions and methods described herein involve expression vectors (e.g., nucleic acids encoding a suicide gene and a Fibroblast Growth Factor 18 (FGF-18) polypeptide or a functional fragment thereof delivered by an adeno-associated virus (AAV)) for treating cartilage disorders. In some embodiments, the cartilage disorder is osteoarthritis. FGF-18 restores cartilage following degradation (e.g.., cartilage degradation in osteoarthritis).

### Suicide Gene

Expression vectors of the invention feature a nucleic acid encoding a suicide gene (e.g., a gene capable of causing apoptosis of the cell carrying the expression vector encoding the suicide gene, a gene that terminates the transcription or translation of the expression vector, itself, or a gene that reduces the expression level of the expression vector by encoding an interfering RNA molecule).

In some embodiments, the invention features a suicide gene that is a gene capable of causing apoptosis of the cell carrying the expression vector encoding the suicide gene. In some embodiments, the invention features a suicide gene chosen from one or more of the genes selected from the list including: adenovirus (ADV) E1A, ADV E4, ADV E4orf6, hepatitis B virus (HBV) HBx, human papillomavirus (HPV) E1\hat{ }E4, HPV E6, HPV E7, poliovirus (PLV) 2Apro, PLV 2B, PLV 3A, PLV 3Cpro, avian encephalomyelitis virus (AEV) 2C, AEV VP3, bovine leukosis virus (BLV) G4, Foot-and-mouth disease virus (FMDV) VP1, hepatitis C virus (HCV) NS3, HCV NS4A, human immunodeficiency virus type (HIV-1) Env, HIV-1 Nef, HIV-1 Protease, HIV-1 Tat, HIV-1 Vpr, human T-cell leukemia virus type 1 (HTLV-1) p13(II), influenza A virus (IAV) PB1-F2, SARS coronavirus (SARS-CoV) 7A, vesicular stomatitis virus (VSV) M, VSV P, walleye dermal sarcoma virus (WDSV) OrfC, West Nile virus (WNV) Capsid, WNV NS2B, WNV NS3, Akt2, Pik3r2, Tnfrsf1a, Pp3cb, Ppp1r13b, Ikbkb, Prkar2a, c-Myc, CHOP, TRAF-2, ATF-4, XBP-1spl, MnSOD, IkBalpha, Hsp70, Hsp26, Fas, CD40, CEBP beta, CEBP delta, D-bind, IL-15, IL-10, MCP-1, ICAM-1, MHC-1 -related genes, Stat-1, IRF-1, IRF-7, c-jun, p53, AP-1, HRK, Fas-L, Bim, Bak, Bax, PIDD, Bid, Apaf-1, TRAILR2, PUMA, NOXA, GZMB, capase 6 (CaspP6), caspase 3 (CaspP-3), caspase 7 (CaspP7), and TRAIL-R. In some embodiments, the suicide gene may be one or more genes related to proteasome components.

In some embodiments, the suicide gene is the cytosine deaminase gene, a gene that encodes the RQR85 polypeptide, or a gene that encodes a truncated human Epidermal Growth Factor receptor polypeptide.

In some embodiments, the suicide gene is a gene that terminates the transcription or translation of the expression vector. In some embodiments, the suicide gene is a nuclease, wherein the nuclease disrupts the transcription or translation of the expression vector. In some embodiments, the nuclease is a clustered regulatory interspaced short palindromic repeat (CRISPR)-associated protein. In some embodiments, the CRISPR-associated protein is CRISPR-associated protein 9. In some embodiments, the CRISPR-associated protein is CRISPR-associated protein 12a. In some embodiments, the nuclease is a transcription activator-like effector nuclease (TALEN), a meganuclease, or a zinc finger nuclease (ZFN). In some embodiments the inducible suicide gene is a guide RNA (gRNA) in a nuclease-mediated gene editing system.

In some embodiments, the suicide gene is a gene that reduces the expression level of the expression vector by encoding an interfering RNA molecule. In some embodiments, the suicide gene is a nucleic acid molecule (e.g., DNA molecule or RNA molecule, e.g., mRNA or inhibitory RNA molecule (e.g., short interfering RNA (siRNA), micro RNA (miRNA), or short hairpin RNA (shRNA)), or a hybrid DNA-RNA molecule).

### I. Inducible Suicide Gene

In some embodiments, the invention features an inducible suicide gene (e.g., a suicide gene activated by a small molecule).

In some embodiments, the inducible suicide gene is an HSV-TK gene (e.g. see Dey, Dilip, and Gregory R D Evans. “Suicide gene therapy by herpes simplex virus-1 thymidine kinase (HSV-TK).” Targets in Gene Therapy (2011): 65.).

In some embodiments, the inducible suicide gene encodes an amino acid sequence that has at least 85% sequence identity (e.g., at least 86%, at least 87%, at least 88%, at least 89%, at least 90%, at least 91%, at least 92%, at least 93%, at least 94, at least 95%, at least 96%, at least 97%, at least 98%, or at least 99% identity) to the amino acid sequence of SEQ ID NO: 1.

In some embodiments, the inducible suicide gene is a rapamycin-activated Casp9 (e.g., see Stavrou, Maria, et al. “A rapamycin-activated caspase 9-based suicide gene.” Molecular Therapy 26.5 (2018): 1266-1276.).

In some embodiments, the inducible suicide gene encodes an amino acid sequence that has at least 85% sequence identity (e.g., at least 86%, at least 87%, at least 88%, at least 89%, at least 90%, at least 91%, at least 92%, at least 93%, at least 94, at least 95%, at least 96%, at least 97%, at least 98%, or at least 99% identity) to the amino acid sequence of SEQ ID NO: 2.

In some embodiments, the invention features an inducible suicide gene system including a thymidine kinase suicide gene, cytosine deaminase/5 fluorocytosine response element, herpes simplex virus/ganciclovir (HSV-tk/GCV) response element, Carboxyl Esterase/Irinotecan, Varicella Zoster Virus Thymidine Kinase/6-Methoxypurine Arabinonucleoside, NitroreductaseNfsb/5-(Aziridin-1-yl)-2 4-Dinitrobenzamide, Carboxypeptidase G2/4-[(2-chloroethyl)(2-mesyloxyethyl) amino]benzoyl-L-glutamic acid, and cytochrome p450-ifosfamide or cytochrome p450-cyclophosphamide.

### II. Nuclease-Mediated Disruption of the Expression Vector

In some embodiments, the suicide gene may be a nuclease or gRNA. Any suitable nuclease may be used. In some embodiments, the suicide gene is a component of a nuclease-mediated gene editing system. For example, the suicide gone introduces an alteration (e.g., insertion, deletion (e.g., knockout), translocation, inversion, single point mutation, or other mutation) in a gene that enables the transcription or translation of the expression vector. Exemplary gene editing systems include the CRISPR system, meganucleases, the ZFNs, and TALENs. CRISPR-based methods, ZFNs, and TALENs are described, e.g., in Gaj et al. Trends Biotechnol.31.7(2013): 397-405.

For example, a useful tool for the disruption and/or integration Of target genes into the genome of a cell is the CRISPR/Cas system, a system that originally evolved as an adaptive defense mechanism in bacteria and archaea against viral infection. The CRISPR/Cas system includes palindromic repeat sequences within plasmid DNA and a Cas protein (e.g., Cas9 or Cas12a). This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host veil to create a gRNA, which can subsequently anneal to a target sequence and localize the Cas nuclease to this site. In this manner, highly site-specific Cas-mediated DNA cleavage can be caused in a foreign polynucleotide because the interaction that brings Cas within close proximity of the target DNA molecule is governed by RNA:DNA hybridization. As a result, one can design a CRISPR/Cas system to cleave a target DNA molecule of interest (e.g., a gene that enables the transcription or translation of the expression vector). This technique has been exploited in order to edit eukaryotic genomes (Hwang et al. Nature Biotechnology 31:227 (2013), the disclosure of which is incorporated herein by reference) and can be used as an efficient means of site-specifically editing cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference,

The CRISPR system has been modified for use n gene editing (e.g., changing, silencing, and/or enhancing certain genes) in eukaryotes. See, e.g., Wiedenheft et al., Nature 482: 331, 2012. For example, such modification of the system includes introducing into eukaryotic net a plasmid containing a specifically designed CRISPR and one or more appropriate Cas proteins. The CRISPR locus is transcribed into RNA and processed by Cas proteins (e.g., Cas9) into small RNAs that contain a repeat sequence flanked by a spacer. The RNAs serve as guides to direct Cas proteins to silence specific DNA/RNA sequences, depending on the spacer sequence. See, e.g., Horvath et at., Science 327: 167, 2010; Makarova et al., Biology Direct 1:7, 2006: Pennisi, Science 341: 833, 2013. In some examples, the CRISPR system includes the Cas9 protein, a nuclease that cuts on both strands of the DNA.

In some embodiments, in a CRISPR system for use described herein, e.g., in accordance with one or more methods described herein, the spacers of the CRISPR are derived from a target gene sequence, e.g., from a sequence of a gene that enables the transcription or translation of the expression vector.

In some embodiments, the suicide gene includes a gRNA for use in a CRISPR system tor gene editing. In some embodiments, the suicide gene contains a meganuclease, or an mRNA encoding a meganuclease, that targets (e.g., cleaves) a nucleic acid sequence (e.g., DNA sequence) of a gene that enables the transcription or translation of the expression vector. In some embodiments, the suicide gene contains a ZFN, or an mRNA encoding a ZFN, that targets (e.g., cleaves) a nucleic acid sequence (e.g., DNA sequence) of a gene that enables the transcription or translation of the expression vector. In some embodiments, the suicide gene contains a TALEN, or an mRNA encoding a TALEN, that targets (e.g., cleaves) a nucleic acid sequence (e.g., DNA sequence) of a gene that enables the transcription or translation of the expression vector. In some embodiments, the suicide, gene contains a Cas (e.g., Cas9), or an mRNA encoding a Cas (e.g., Cas9), that targets (e.g., cleaves) a nucleic, acid sequence (e.g., DNA sequence) of a gene that enables the transcription or translation of the expression vector.

In certain embodiments, the CRISPR system is used to edit (e.g., to add or delete a base pair) a target gene, e.g., a gene that enables the transcription or translation of the expression vector. In other embodiments, the CRISPR system is used to introduce a premature stop codon, e.g., thereby decreasing the expression of a target gene. In yet other embodiments, the CRISPR system is used to turn off a target gene Era a reversible manner, e.g., similarly to RNA interference. In some embodiments, the CRISPR system is used to direct Cas (e.g., Cas9) to a promoter of a target gene, e.g., a gene That enables the transcription or translation of the expression vector, thereby blocking an RNA polymerase sterically.

In some embodiments, a CRISPR system can be generated to edit a gene that enables the transcription or translation of the expression vector using technology described in, e.g., U.S. Publication No. 20140068797; Cong, Science 339; 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., a gene that enables the transcription or translation of the expression vector. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-5 gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene that inhibits a gene that enables the transcription or translation of the expression vector. In the CRISPRa technique, dCas9 fusion proteins recruit transcriptional activators. For example, dCas9 can be used to recruit polypeptides (e.g., activation domains) such VP 64 or the p65 activation domain (p65D) and used with sgRNA (e.g., single sgRNA or multiple sgRNAs), to activate a gene or genes, e.g., endogenous gene(s). Multiple activators can be recruited by using multiple sgRNAs, which can increase activation efficiency. A variety of activation domains and single or multiple activation domains can be used. In addition to engineering dCas9 to recruit activators, sgRNAs can also be engineered to recruit activators. For example RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Biol. 17; 5, 2016, incorporated herein by reference.

In some embodiments, the gRNA or Cas (e.g., Cas9) can be used in a CRISPR system to engineer an alteration in a gene (e.g., a cleric that enables the transcription or translation of the expression vector). In other examples, the meganuclease, ZFN, and/or TALEN can be used to engineer an alteration in a gene (e.g., a gene that enables the transcription or translation of the expression vector). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) a gene that enables the transcription or translation of the expression vector, e.g., the alteration is a negative regulator of function. In yet another example, the alteration elicits a defect (e.g., a mutation causing a defect), in a gene that enables the transcription or translation of the expression vector.

Alternative methods for disruption of a target DNA by site-specifically cleaving genomic DNA prior to the incorporation of a gene of interest in a cell include the use of meganucleases, ZFNs, and TALENs. Unlike the CRISPR/Cas system, these enzymes do not contain a guiding polynucleotide to localize to a specific target sequence. Target specificity is instead controlled by DNA binding domains within these enzymes. The use of meganucleases, ZFNs, and TALENs genome editing applications is described, e.g., in Umov et al. Nature Reviews Genetics 11:636 (2010); and in Joung et al. Nature Reviews Molecular Cell Biology 14:49 (2013), the disclosure of both of which are incorporated herein by reference. In some embodiments, a gene that enables the transcription or translation of the expression vector may be disrupted in the cells containing the expression vector using these gene editing techniques described herein.

III. Suicide Genes that Reduce the Expression Level of the Expression Vector by Encoding an Interfering RNA Molecule

In some embodiments, the suicide gene is an inhibitory RNA molecule, e.g., that acts by way of the RNA interference (RNAi) pathway. An inhibitory RNA molecule can decrease the expression level (e.g., protein level or mRNA level) of a gene that enables the transcription or translation of the expression vector. For example, an inhibitory RNA molecule includes a siRNA, shRNA, and/or a miRNA that targets a gene that enables the transcription or translation of the expression vector. An siRNA is a double-stranded RNA molecule that typically has a length of about 19-25 base pairs. An shRNA is an RNA molecule containing a hairpin turn that decreases expression of target genes via RNAi. shRNAs can be delivered to cells in the form of plasmids (e.g., viral or bacterial vectors), by transfection, electroporation, or transduction. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. In some embodiments, the inhibitory RNA molecule decreases the level and/or activity of function of a gene that enables the transcription or translation of the expression vector function. In other embodiments, the inhibitory RNA molecule decreases the level and/or activity of an inhibitor of a positive regulator of function.

An inhibitory RNA molecule can be modified, e.g., to contain modified nucleotides, e.g., 2′-fluoro, 2-o-methyl, 2′-deoxy, unlocked nucleic acid, 2′-hydroxy, phosphorothioate, 2′-thiouridine, 4′-thiouridine, or 2′-deoxyuridine. Without being bound by theory, it is believed that certain modification can increase nuclease resistance and/or serum stability or decrease immunogenicity.

In some embodiments, the inhibitory RNA molecule decreases the level and/or activity or function of a gene that enables the transcription or translation of the expression vector. In some embodiments, the inhibitory RNA molecule inhibits expression of a gene that enables the transcription or translation of the expression vector. In other embodiments, the inhibitory RNA molecule increases degradation a gene that enables the transcription or translation of the expression vector. The inhibitory RNA molecule can be chemically synthesized or transcribed in vitro. The making and use of inhibitory therapeutic agents based on non-coding RNA such as ribozymes, RNAase P, siRNAs, and miRNAs are also known in the art, for example, as described in Sioud, RNA Therapeutics: Function, Design, and Delivery (Methods in Molecular Biology). Humana Press 2010.

### Safety Element

In some embodiments, the invention features a nucleic acid encoding a safety element (e.g., a gene capable of causing suppression of expression, suppression of transcription, suppression of translation, attenuation of efficacy, senescence of the cell carrying the construct, or apoptosis of the cell carrying the expression vector).

In some embodiments, the invention features a safety element that can transcribe an interfering RNA molecule, such as a short interfering RNA, micro RNA, or short hairpin RNA; or a long non-coding RNA designed to interfere with FGF-18 mRNA via the RNA-induced silencing complex, Drosha-, or Dicermediated pathways, or simply via sequestration or destabilization of the FGF-18 mRNA; or a ribozyme-mediated system for the degradation of FGF-18.

In some embodiments, the invention features a safety element that can encode repressor proteins specific to the nucleic acid encoding FGF-18 or a functional fragment thereof.

In some embodiments, the invention features a safety element that can encode general repressor proteins to facilitate cellular senescence.

In some embodiments, the invention features a safety switch that is one or more general repressors selected from: a gene encoding one or more of the polycomb proteins, a gene encoding one or more of the PRE TF binding elements, a gene encoding NRR (EAR-r), a gene encoding AtERF4, a gene encoding HAD-19, or a gene encoding one or more of the histone deacetylase family of proteins.

### Fibroblast Growth Factor 18 Polypeptide or a Functional Fragment Thereof

Expression vectors of the invention feature a nucleic acid encoding a Fibroblast Growth Factor 18 (FGF-18) polypeptide or a functional fragment thereof.

In some embodiments, the expression vector encodes a FGF-18 polypeptide or functional fragment thereof, wherein the FGF-18 polypeptide has at least 85% sequence identity (e.g., at least 86%, at least 87%, at least 83%, at least 89%, at least 90%, at least 91%, at least 92%, at least 93%, at least 94, at least 95%, at least 96%, at least 97%, at least 98%, or at least 99% identity) to the amino acid sequence of SEQ ID NO: 3.

In some embodiments, the expression vector encodes an FGF-18 polypeptide that may be in its native form, in its mature form, in a recombinant form, or any naturally occurring variants of FGF-18 (e.g., splice variants or allelic variants).

In some embodiments, the expression vector encodes a FGF-18 polypeptide or variant of post-translational modification thereof, wherein the FGF-18 polypeptide includes one or more of the naturally occurring post-translational modifications that occur in an FGF-18 post-translational modification variant.

### Transcriptional Regulator

In some embodiments, expression vectors of the invention feature a transcriptional regulator operably linked to a nucleic acid sequence encoding FGF-18 or a functional fragment thereof that functions in response to a stimulus (e.g., an internal stimuli, inflammation, cytokines, low levels of extracellular growth factors, infection, hypoxia, low or high levels of iron, low or high levels of ferritin, low or high levels of ampicillin, low or high levels of isopropyl β-d-1-thiogalactopyranoside, doxycycline, nitric oxide, nitric oxide synthases (NOS); paracrine, endocrine, or autocrine signaling; external stimuli, or external stimuli that cause tissue degeneration or infection that causes structural changes in the proximity of the cell carrying the expression vector).

In some embodiments, the transcriptional regulator is one or more of the elements selected from the list including: a promoter, a repressor, a TATA box, a B recognition element recognition element, a transcription factor II B recognition element, and a downstream promoter element.

In some embodiments, the invention features a transcriptional regulator is one or more of the promoters selected from the list including: a lacUV5 promoter, an FGF gene family promoter, a transforming growth factor gene family promoter, a Hyaluronan synthase gene family promoter, a promoter responding to inflammation, a promoter that is inducible by cytokines, a tac promoter, a trc promoter, a Salmonella enterica prpBCDE (prpB) promoter, an isocitrate lyase (aceA)/malete synthase (aceB) promoter, a gluconate permease (gntP)/gluccnokinase (gntK) promoter, a CJ10x promoter, a tac-M promoter, a maltose ABC transporter periplasmic protein (malE1) promoter, a ARF GTPase-activating protein GIT1 (git1) promoter, a BCL-2 associated agonist of cell death (BAD) promoter, a Squamosa Promoter-binding protein-like transcription factor (SPLs), a 4-N14 promoter, a hybrid interleukin 1 enhancer/interleukin 6 promoter, a prostaglandin-endoperoxide synthase 2 (COX-2) promoter, an inducible cytokine promoter, a C-X-C motif chemokine ligand 10 (CXCL10) promoter, a Lac/LacUV5 promoter, a T7 promoter, a NOS promoter, a ubiquitin 1 promoter, a ribulose-1,5-bisphosphate carboxylaseioxygenase (rbcS) promoter, an arabinose-inducible araBAD promoter (araPBAD), an Escherichia coli rharrinose BAD promoter (rhaPBAD), a phage lambda pL/pR promoter, an Escherichia coli cold shock protein A (cspA) promoter, a cauliflower mosaic virus promoter, a cauliflower mosaic virus 35S promoter, a RNA small regulatory RNA antitoxin SokC (SokC) promoter, an uncharacterized lipoprotein YafT (yaft) promoter, a putative integrase (pintF) promoter, an Anhydromuropeptide permease (ampG) promoter, an acid (poly)phosphatase (appY) promoter, a Lipase a, lysosomal acid type (lipA) promoter, a cytomegalovirus (CMV) early promoter, an elongation factor-1 (EF1) alpha promoter, an EF1 promoter, a CAG promoter, a Herpes Simplex Virus-1 Thymidine Kinase promoter, a human beta-actin promoter, a simian vacuolating virus 40 (SV40) promoter, a murine stem cell virus promoter, a phosphoglycerate kinase (PGK) promoter, a ubiquitin C promoter, or any other known constitutive or inducible promoter.

In some embodiments, the promoter is a PGK promoter.

In some embodiments, the promoter is a CMV promoter.

### Additional Components or Modifications

It should be understood that genes can be used with or without introns (e.g., naturally occurring introns, minute virus of mice introns, or synthetic introns), or/or with only a subset of the introns (e.g., naturally occurring introns, minute virus of mice introns, or synthetic introns), and/or with codon optimization of the nucleic sequence in the expression vector.

In some embodiments, the nucleic acid encoding an FGF-18 polypeptide or a functional fragment thereof may additionally include one or more secretion sequences to ensure that the polypeptide is targeted to the appropriate cellular compartment or extracellular space.

In some embodiments, the secretion sequence is one or more of the amino acid sequences selected from the list including:

| Amino acid sequence | SEQ ID NO: |
|---|---|
| SWSHPQFEK | 5 |
| GCKPCICTVPEVSSVFIF | 6 |
| PPKPKDV | |
| ICTVPEVSSVFIFPPKPKDV | 7 |
| KPCICTVPEVSSVFIFPPKPKDV | 8 |
| SVFIFPPKPKDV | 9 |
| PPKPKDV | 10 |
| PCICTVPEVSSVFIEFPPKPKDV | 11 |
| MWWRLWWLLLLLLLLWPMVWA | 12 |
| METDTLLLWVLLLWVPGSTG | 13 |
| MDMRVPAQLLGLLLLWVLRGARC | 14 |
| MPLLLLLPLLWAGALA | 15 |
| MDAMKRGLCCVLLLCGAVFVSPS | 16 |
| MLLLLLLLLLLALALA | 17 |
| MLLLLLLLGLRLQLSLG | 18 |
| MGVKVLFALICIAVAEA | 19 |
| MKWVTFISLLFLFSSAYS | 20 |
| MAFLWLLSCWALLGTTFG | 21 |
| MQLLSCIALILALV | 22 |
| MNLLLILTFVAAAVA | 23 |
| MALWMRLLPLLALLALWGPD | 24 |
| PAAAF | |
| MYSAPSACTCLCLHFLLLC | 25 |
| FQVQVLVA | |

In some embodiments, the expression vector may also include an enhancer.

In some embodiments, the expression vector may also include a promoter.

In some embodiments, the promoter is a constitutive promoter.

In some embodiments, the promoter is an inducible promoter.

In some embodiments, the inducible promoter is one or more of the inducible promoters selected from the list including: lacUV5, tac, trc, prpB, aceA/aceB, gntP/gntK, CJ10x2, tac-M, malE1, git1, BAD, SPLs, 4-N14, CXCL10, Lac/LacUV5, T7, araPBAD, rhaPBAD, pL/pR, cspA, COX-2, a hybrid IL-1 enhancer/IL-6 promoter, a CASI promoter (e.g., a synthetic promoter that includes CMV and UbC enhancer elements and the chicken b-actin promoter), a collagen type II promoter, or a cytokine inducible promoter.

In some embodiments, the promoter is responsive to a stimulus that is orthogonal, semi-orthogonal, and/or opposing to the transcription regulator.

In some embodiments, the expression vector may also include one or more silencers that respond to a stimulus that is orthogonal, semi-orthogonal, and/or opposing to the suicide gene.

In some embodiments, the silencer may be one or more of the silencers selected from the gene list including: CBX, Bcl6, E2F6, PDGFA 5′ SHS, T39, Jarid2, REST, YY1, PRE-2-S5, Gal4-CBX4, Myh6 PNR, ZEBB, Gfi1/1b, Elk-3, ETV6/7, Stat5, Gata3, Runx1, Sp1/Klf, Xist, COOLAIR or HOTAIR, Meg3, or Fendrr.

In some embodiments, the expression vector may also include a poly adenylation signal (e.g., a naturally occurring poly adenylation signal, a bovine growth hormone poly adenylation signal, or a synthetic poly adenylation signal).

In some embodiments, the poly adenylation sional is a general mammalian poly adenylation consensus sequence or a variation thereof, including a U-rich stretch, followed by approximately 0 to 20 nucleotides (e.g., 1 to 19 nucleotides, 2 to 18 nucleotides, 3 to 17 nucleotides, 4 to 16 nucleotides, 5 to 15 nucleotides. 6 to 14 nucleotides, 7 to 13 nucleotides, 8 to 12 nucleotides, 9 to 11 nucleotides, or about 10 nucleotides), followed by a semi-conserved consensus sequence of ATAAA, followed by approximately 15 to 30 (e.g., 16 to 29 nucleotides, 17 to 28 nucleotides, 18 to 27 nucleotides, 19 to 26 nucleotides, 20 to 25 nucleotides, 21 to 24 nucleotides, or 22 to 23 nucleotides) nucleotides, followed by cytosine (C)A, followed by 0 to 20 nucleotides (e.g., 1 to 19 nucleotides, 2 to 18 nucleotides, 3 to 17 nucleotides, 4 to 16 nucleotides, 5 to 15 nucleotides, 6 to 14 nucleotides, 7 to 13 nucleotides, 8 to 12 nucleotides, 9 to 11 nucleotides, or about 10 nucleotides) followed by a differentiation-specific element, a G or U, or a U-rich stretch.

In some embodiments, the expression vector may also include a termination region.

In some embodiments, the termination sequences may be one or more of the stop codons or termination sequences selected from the list of nucleic acids encoding a stop codon or termination including: TAG, TAA, TGA, or TTTATT.

In some embodiments, the expression vector may also include one or more alternative splicing exons and/nitrons, proximal control elements, and/or downstream sequences.

In some embodiments, the expression vector may also include one or more of a cis-regulatory module. In some embodiments, the expression vector may also include a Woodchuck Hepatitis Virus (WHP) Posttranscriptional Regulatory Element (WPRE).

### Delivery

Any suitable expression vector may be used in conjunction with the present compositions and methods to design and assemble the components of the nucleic acid encoding a suicide gene and a FGF-18 polypeptide or a functional fragment thereof. In one embodiment, the expression vector is a viral vector carrying the nucleic acid encoding a suicide gene, and a FGF-18 polypeptide or a functional fragment thereof. Methods for assembly of the recombinant vectors are known in the art. See, e.g., Griffiths, A. J., et al. “Making recombinant DNA,” An Introduction to Genetic Analysis. 7th edition. New York: WH Freeman. Available from: http://www.ncbi.nlm.nih.gov/books/NBK21881 (2000).

### I. Viral Vectors for Expression of a Therapeutic Nucleic Acids

Viral genomes provide a rich source of vectors that can be used for the efficient delivery of exogenous genes into a mammalian cell (e.g., chondrocytes, chondrocyte progenitor cells, and synoviocyte cells). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34,Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncioretroviruses, HTLV-BLV group, lentivirus, alpharetmvirus, gammaretrovirus, spumavirus (Coffin, J. M., Retroviridae: The viruses and their replication, Virology, Third Edition (Lippincott-Raven, Philadelphia, (1996)). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### IA. Viral Capsid Proteins

The nucleic acids and vectors described herein can be incorporated into a recombinant virion in order to facilitate introduction of the nucleic acid or vector into a cell. In some embodiments, the viral capsid protein is a naturally occurring capsid or an engineered capsid protein (e.g., a capsid that contains a modified sequence).

The capsid proteins of the viral vector compose the exterior, non-nucleic acid portion of the virion and are encoded by the nucleic acid. In one embodiment, the viral capsid proteins may be structurally composed from one or more in the following list: an AAV capsid protein including a natural and/or a synthetic serotype, a Lentiviral capsid protein of one or more serotypes, a Retroviral capsid protein of one or more serotypes, a Herpesviridae capsid protein of one or more serotypes, an adenoviridae capsid protein with one or more serotypes, a parvoviridae capsid protein with one or more serotypes, a rhabdoviridae capsid protein with one or more serotypes, a reoviridae capsid protein with one or more serotypes, a orthomyxoviridae capsid protein with one or more serotypes, a bunyaviridae capsid protein with one or more serotypes, a flaviviridae capsid protein with one or more serotypes, a retroviridae capsid protein with one or more serotypes, a paramoxyviridae capsid protein with one or more serotypes, a potyviridae capsid protein with one or more serotypes, a coronaviridae capsid protein with one or more serotypes, a caliciviridae capsid protein with one or more serotypes, a papillomaviridae capsid protein with one or more serotypes, a poxviridae capsid protein with one or more serotypes, a polymaviridae capsid protein with one or more serotypes, or any other known virus or viroid capsid proteins with one or more serotypes.

In some embodiments, a naturally occurring viral capsid protein is modified (e.g., by conjugation for the expression of non-viral peptides on the surface of a naturally occurring viral capsid proteins for cell type specific targeting, or by conjugation for the expression of fusion proteins between the viral capsid protein, eukaryotic proteins, or protein fragments for the narrowing of tropism).

In some embodiments, the engineered viral capsid protein is conjugated to a ligand (e.g., for cell type specificity or modulation of immunogenicity).

### IB. Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral complimentary DNA (cDNA) deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (VVPRE), as described in U.S. Pat. No. 6,136,597, the disclosure of which is incorporated herein by reference as it pertains to WPRE. The lentiviral vector may further include a backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods arid compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther. 8:811 (2001), Osborn et al, Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may be a clinical grade vector.

### IC. Adeno-Associated Viral Vectors

Nucleic acids of the compositions and methods described herein may be incorporated into recombinant adeno-associated virus (rAAV) vectors and/or virions in order to facilitate their introduction into a cell (e.g., chondrocyte, chondrocyte precursor, and synoviocyte cells). AAV vectors can be used in the central nervous system, and appropriate promoters and serotypes are discussed in Pignataro et al., J Neural Transm., 125: 575 (2018), the disclosure of which is incorporated herein by reference as it pertains to promoters and AAV serotypes useful in CNS gene therapy. An AAV promoter used in the compositions and methods described herein may include one or more of a phosphoglycerate kinase promoter or a cytomegalovirus promoter. rAAV vectors useful in the compositions and methods described herein are recombinant nucleic acid constructs (e.g., nucleic acids capable of expression in chondrocyte, chondrocyte precursor, and synoviocyte cells) that include (1) a heterologous sequence to be expressed and (2) viral sequences that facilitate integration and expression of the heterologous genes. The viral sequences may include those sequences of AAV that are required in cis for replication and packaging (e.g., functional inverted terminal repeat sequences (ITR)) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

### ICI. Adeno-Associated Viral Vector Capsid Protein

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305: 6,057,152; and 6,376,237; as well as in Rabinowitz et al., J. Viral, 76:791 (2002) and Bowles et al., J. Viral. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4. 5, 6. 7, 8, 9, 10 and rh74. Construction and use of AAV vectors and AAV proteins of different serotypes are described, for example, in Chao et al,, Mol, Ther. 2:619 (2000); Davidson et al., Proc. Natl. Acad. Sci. USA 97:3428 (2000); Xiao et al., J. Virol. 72:2224 (1998); Halbert et al., J. Viral. 74:1524 (2000); Halbert et al., J. Virol. 75:6615 (2001); and Auricchio et al., Hum. Molec, Genet, 10:3075 (2001), the disclosures of each of which are incorporated herein by reference as they pertain to AAV vectors for oene delivery.

In some embodiments, the viral vector is an AAV2.

In some embodiments, the viral vector is an AAV5.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec, Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV ViriOnS that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

Desirable AAV fragments for assembly into expression vectors include the cap proteins, including the vp1, vp2, vp3, and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is utilized with the ITRs from an AAV having a different capsid protein, are useful in the invention.

In some embodiments, the viral capsid protein includes a ligated derivative (e.g., a synthetic, a biological, or a semi-synthetic derivative).

### II. Methods for the Delivery of Exogenous Nucleic Acids to Target Cells

Techniques that can be used to introduce a polynucleotide, such as DNA or RNA, including codon-optimized DNA or RNA, into a mammalian cell (e.g., chondrocyte, chondrocyte precursor, and synoviocyte cells) are well known in the art. For example, electroporation can be used to permeabilize mammalian cells (e.g., human target cells) by the application of an electrostatic potential to the cell of interest. Mammalian cells, such as human cells, subjected to an external electric field in this mariner are subsequently predisposed to the uptake of exogenous nucleic acids (e.g., nucleic acids capable of expression in e.g., chondrocyte, chondrocyte precursor, and synoviocyte cells). Electroporation of mammalian cells is described in detail, e.g., in Chu et al., Nucleic Acids Research 15:1311 (1987), the disclosure of which is incorporated herein by reference. A similar technique, NUCLEOFECTION™ utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and DEAE-dextran, the use of which as a transfection agent is described in detail, for example, in Gulick et al., Current Protocols in Molecular Biology 40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, sIRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

Exosomes, extracellular vesicles, minicircle DNA, nanoplasmid™, ministring DNA, doggybone DNA, and closed-end DNA represent other potential vehicles that can be used to modify the genome of a target cell according to the methods described herein.

In some embodiments, the expression vector includes a nucleic acid sequence that includes a modified nuclear targeting sequence.

In some embodiments, the expression vector includes a nucleic acid sequence that is circularized (e.g., for efficient expression).

### IIA. Non-Viral Particle

In some embodiments, the invention features an expression vector that includes a non-viral particle. In some embodiments, the non-viral particle is any one or more selected from the group including: a lipid nanoparticle, a lipid envelope, a phospholipid bilayer, a liposome, a noisome, a fullerene, a protein-based nanoparticle, a nanocrystal, a dendrimer, an organic/inorganic colloid, a pegylated liposome, a polymeric micelle, a reverse micelle, a mixed micelle, a sensitive micelle, a multicomponent micelle, or a poly- or uni-molecular dendritic micelle.

In some embodiments, the invention features an expression vector that includes a non-viral particle that is ligated to one or more chemical moieties for cell type-specific targeting, or cellular or tissue tropism optimization.

### IIB. Viral Capsid Proteins

In some embodiments, the invention features an expression vector that includes a viral capsid protein in order to facilitate introduction of the nucleic acid or vector into a cell. In some embodiments, the viral capsid protein is a naturally occurring capsid or an engineered capsid protein (e.g., capsids that contain modified sequences).

The capsid proteins of the viral vector compose the exterior, non-nucleic acid portion of the virion and are encoded by the nucleic acid. In one embodiment, the viral capsid proteins may be structurally composed from one or more in the following list: an AAV capsid protein including a natural and/or a synthetic serotype, a Lentiviral capsid protein of one or more serotypes, a Retroviral capsid protein of one or more serotypes, a Herpesviridae capsid protein of one or more serotypes, an adenoviridae capsid protein with one or more serotypes, a parvoviridae capsid protein with one or more serotypes, a rhabdoviridae capsid protein with one or more serotypes, a reoviridae capsid protein with one or more serotypes, a orthomyxoviridae capsid protein with one or more serotypes, a bunyaviridae capsid protein with one or more serotypes, a flaviviridae capsid protein with one or more serotypes, a retroviridae capsid protein with one or more serotypes, a paramoxyviridae capsid protein with one or more serotypes, a potyviridae capsid protein with one or more serotypes, a coronaviridae capsid protein with one or more serotypes, a caliciviridae capsid protein with one or more serotypes, a papillomaviridae capsid protein with one or more serotypes, a poxviridae capsid protein with one or more serotypes, a polymaviridae capsid protein with one or more serotypes, or any other known virus or viroid capsid proteins with one or more serotypes.

In some embodiments, a naturally occurring viral capsid protein is modified (e.g., by conjugation for the expression of non-viral peptides on the surface of a naturally occurring viral capsid proteins for cell type specific targeting, or by conjugation for the expression of fusion proteins between the viral capsid protein, eukaryotic proteins, or protein fragments for the narrowing of tropism).

In some embodiments, the engineered viral capsid protein is conjugated to a ligand (e.g., for cell type specificity or modulation of immunogenicity).

### III. Exemplary Expression Vectors for Expression of a Therapeutic Nucleic Acids

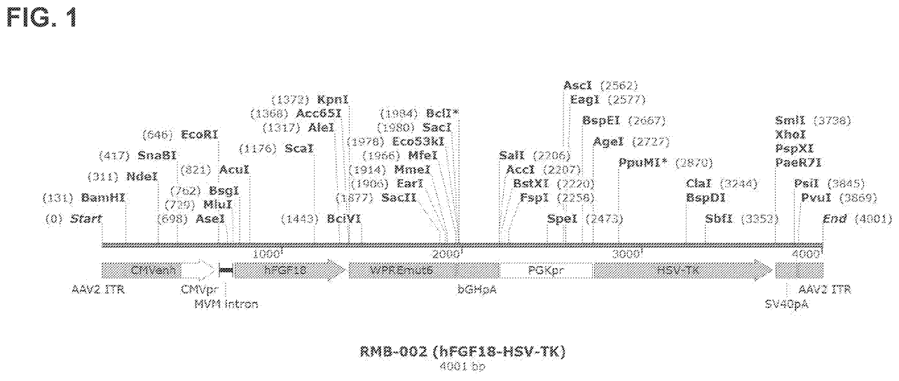

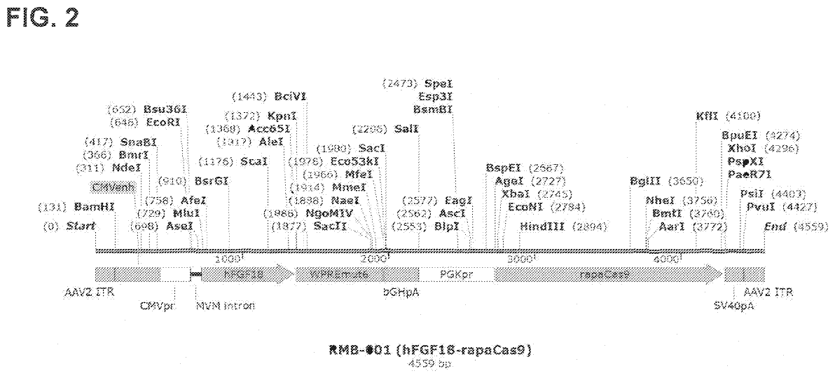

In some embodiments, the expression vector is a viral vector (e.g., AAV2) including a nucleic acid encoding a suicide gene, and FGF-18 polypeptide or a functional fragment thereof. For example, the viral vector may include the nucleic acid sequence of a cytomegalovirus (CMV)-enhancer (CMVenh) operatively linked to a human FGF18 (hFGF18) polypeptide, a woodchuck hepatitis virus post-transcriptional regulatory element mutant 6 (WPREmut6; a DNA element known to substantially increase expression levels but without promoter activity), a bovine growth hormone polyadenylation signal (bGHpA), a HSV-TK suicide gene, a mutated SV40 early polyadenylation signal (SV40pA), and flanking AAV2 inverted terminal repeat sequences (ITR) (FIG. 1). Alternatively, for example, the viral vector may include the nucleic acid sequence of a CMVenh operatively linked to a hFGF18 polypeptide, a WPREmut6, a bGHpA, a rapaCas9 suicide gene, a SV40pA, and flanking AAV2 ITR (FIG. 2).

In some embodiments, the expression vector is modified (e.g., to remove the ITR and place the expression vector in a minicircle or any other configuration that can be targeted optimally to the nucleus for efficient expression with and without integration).

### IV. Delivery Devices for the Delivery of Therapeutic Nucleic Acids

The present invention may be delivered without limitation via a drug delivery device such as a gene-gun, prefilled syringe, autoinjector, aerosol spray, spray cannister, or patch injector, or may be stored in an ampoule, cartridge, capsule, vial, form-fill-seal, or blow-fill-seal container for administration via a secondary suitable means thereafter.

The delivery may be performed to target tissues, periphery, or distal tissues or organs. As such, the construct may be expressed in the cell that is the target of the expressed protein or enzyme in an autocrine matter. Alternatively, the construct may be expressed by cells in the periphery, surrounding, neighboring, or intermixed with the cells and tissues that are the final target of the expressed protein. Finally, the construct may be expressed by cells outside of the target tissue or organ and act in an endocrine matter on one or more target tissues, organs, or cells. Specifically, without limiting the scope of the present invention the construct may be delivered to the synovial joint and may target chondrocytes for expression of FGF-18 and delivery of FGF-18 to neighboring chondrocytes, or to the cells lining the joint capsule for expression of FGF-18 and delivery of FGF-18 to the proximal chondrocytes.

### Formulation and Dosing of Pharmaceutical Compositions

The expression vector (e.g., nucleic acid) described herein can be formulated into pharmaceutical compositions for administration to a patient, such as a human patient exhibiting or at risk of developing a cartilage disorder, in a biologically compatible form suitable for administration in vivo. A pharmaceutical composition containing, for example, an expression vector described herein, such as an AAV2 or an AAV5 comprising a nucleic acid encoding a suicide gene and a FGF-18 polypeptide or a functional fragment thereof, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., a buffer (e.g., phosphate-buffered saline (PBS) 2-(N-morpholino)ethanesulfonic acid (MES), 2,2-Bis(hydroxymethyl)-2,2′,2″-nitrilotriethanol (BlS-TRIS), diglycine (Gly-Gly), saline, 2-Amino-2-methyl-1-propanol (AMP), or N-(2-Hydroxy-1,1-bis(hydroxymethyl)ethyl)glycine (tricine)) and a nucleic acid. The buffer is typically a pharmaceutical grade buffer.

In certain embodiments, pharmaceutical compositions include one or more expression vectors and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, expression vectors may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including an expression vector encompass any pharmaceutically acceptable salts of the inhibitor, esters of the inhibitor, or salts of such esters. In certain embodiments, pharmaceutical compositions including an expression vector, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to an expression vector, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ ((x)-sorbitan mono-9-octadecenoate poly(oxy-1,2-ethanediyl)) and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™ ((x)-sorbitan mono-9-octadecenoate poly(oxy-1,2-ethanediyl)); the fraction size of polyethylene glycol may be varied; other biocompatible polymers may replace polyethylene glycol, e.g., polyvinyl pyrrolidone: and other sugars or polysaccharides may substitute tor dextrose.

In certain embodiments, a pharmaceutical composition is prepared for administration by injection (e.g., intravenous, intra-articular, sub-chondral, intra-synovial, or intrachondral). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

The dose of the pharmaceutical composition administered to a subject for the treatment of a cartilage disorder (e.g., osteoarthritis) as described herein may depend, for example, on the morphology and or expression profile of hyaline cartilage, the subject, pharmaceutical formulation methods, administration methods (e.g., administration time), the subject's age, body weight, sex, severity of the cartilage disorder being treated, and whether or not the subject has been treated with combination therapies. The dose of the pharmaceutical composition administered may be, for example, from 0.1 mg to 1.0 mg (e.g., from 0.2 to 0.9 mg, from 0.3 to 0.8 mg, from 0.4 to 0.6 mg, or about 0.5 mg). The pharmaceutical composition may be administered in any suitable dosage. Non-limiting examples of dosages are about 0.3 mg of the pharmaceutical composition to about 0.7 mg of the pharmaceutical composition (e.g., from about 0.31 mg of the pharmaceutical composition to about 0.69 mg of the pharmaceutical composition, from about 0.33 mg of the pharmaceutical composition to about 0.67 mg of the pharmaceutical composition, from about 0.38 mg of the pharmaceutical composition to about 0.62 mg of the pharmaceutical composition, from about 0.48 mg of the pharmaceutical composition to about 0.58 mg of the pharmaceutical composition, or about 0.53 mg mg of the pharmaceutical composition). Additional exemplary dosages are about 0.1 mg of the pharmaceutical composition to about 3.0 mg of the pharmaceutical composition (e.g., from about 0.2 mg of the pharmaceutical composition to about 2.9 mg of the pharmaceutical composition, from about 0.4 mg of the pharmaceutical composition to about 2.7 mg of the pharmaceutical composition, from about 0.9 mg of the pharmaceutical composition to about 2.2 mg of the pharmaceutical composition, from about 1.4 mg of the pharmaceutical composition to about 1.7 mg of the pharmaceutical composition, or about 1.55 mg of the pharmaceutical composition).

The pharmaceutical composition may be administered in any suitable dosage such that the number of viral particles delivered is about 1.0×10⁹ vg/mL to about 4.5×10¹¹ vg/mL (e.g., about 1.1×10⁹ vg/mL to about 4.4×10¹¹ vg/mL, about 1.5×10⁹ vg/mL to about 4.0×10¹¹ vg/mL, about 2.5×10⁹ vg/mL to about 3.0×10¹¹ vg/mL, or about 9.1×10¹⁰ vg/mL).

The compositions described herein can be administered in an amount sufficient to improve one or more pathological features in the cartilage disorder (e.g. osteoarthritis). Administration of the compositions described herein may improve the chondrocyte morphology and/or expression profile to more closely resemble the morphology and/or expression profile, respectively, of extracellular matrix associated with hyaline cartilage; improve the synoviocyte morphology and/or expression profile to more closely resemble the morphology and/or expression profile, respectively, of extracellular matrix associated with hyaline cartilage; increase chondrocyte proliferation, increase chondrocyte precursor proliferation, increase cartilage thickness, or increase the total femorotibial joint cartilage thickness. The level of FGF-18 may be assessed to compare the level of the FGF-18 gene and/or protein in subjects before and after treatment in a tissue sample using standard techniques, e.g., enzyme-linked immunoassay, western blot analysis, immunohistochemical analyses, or quantitative reverse transcription polymerase chain reaction. Depending on the outcome of the evaluation, the subject may receive additional treatments.

### Kits

The compositions described herein can be provided in a kit for use in treating a cartilage disorder. The kit may include one or more expression vectors or pharmaceutical compositions as described herein. The kit can include a package insert that instructs a user of the kit, such as a physician, to perform any one of the methods described herein. The kit may optionally include a syringe or other device for administering the composition. The kit may optionally include a carton, a tamper evident seal, a needle, or a prefilled syringe. In some embodiments, the kit may include one or more additional therapeutic agents.

### Combination Therapies

An expression vector described herein can be administered in combination with a one or more additional therapeutic agents for treatment of cartilage disorders. The one or more additional therapeutic agents may include a nonsteroidal anti-inflammatory agent (e.g., piroxicam, ibuprofen, celecoxib, aspirin, etodolac, meloxicam, diclofenac, indomethacin, and naproxen), an analgesic (e.g., capsaicin and acetaminophen), a dietary supplement (e.g., s-adenosyl methionine and hyaluronic acid), or narcotic agent (e.g., tramadol), or a combination thereof.

### EXAMPLES

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1: Treating a Cartilage Disorder in Human Subjects Using a Viral Vector

According to the methods disclosed herein, a subject, such as a human subject, can be administered a pharmaceutical composition (e.g., an AAV2 or an AAV5 viral vector including a nucleic acid encoding a suicide gene and a FGF-18 polypeptide or a functional fragment thereof and a pharmaceutically acceptable carrier, diluent, or excipient; FIGS. 1 and 2) to treat a cartilage disorder in the subject. To this end, the patient is administered a pharmaceutical composition (e.g., an AAV2 or an AAV5 viral vector including a nucleic acid encoding a suicide gene and a FGF-18 polypeptide or a functional fragment thereof and a pharmaceutically acceptable carrier, diluent, or excipient). The pharmaceutical composition thereof is administered to the subject intravenously, intra-articularly, sub-chondrally, intra-synovially, or intrachondrally. The pharmaceutical composition thereof may be administered intravenously, intra-articularly, sub-chondrally, intra-synovially, or intrachondrally and bilaterally to the synovial joint. The pharmaceutical composition may be administered in any suitable dosage. Non-limiting examples of dosages are about 0.1 mg to 0.5 mg, such as 0.1 mg, 0.2 mg, 0.3 mg, 0.4 mg, and 0.5 mg of the pharmaceutical composition. The subject may receive a dosage of about 0.5 mg to about 1.0 mg of the pharmaceutical composition, for example the subject may receive a dosage of about 1.0 mg of the pharmaceutical composition.

### Example 2: Treating a Cartilage Disorder in Human Subjects Using a Lentiviral Vector

According to the methods disclosed herein, a subject, such as a human subject, can be administered a pharmaceutical composition (e.g., a lentivirus viral vector including a nucleic acid encoding a suicide gene and a FGF-18 polypeptide or a functional fragment thereof and a pharmaceutically acceptable carrier, diluent, or excipient) to treat a cartilage disorder in the subject. To this end, the patient is administered a pharmaceutical composition (e.g., a lentivirus viral vector including a nucleic acid encoding a suicide gene and a FGF-18 polypeptide or a functional fragment thereof and a pharmaceutically acceptable carrier, diluent, or excipient). The pharmaceutical composition thereof is administered to the subject intravenously, intra-articularly, sub-chondrally, intra-synovially, or intrachondrally. The pharmaceutical composition thereof may be administered intravenously, intra-articularly, sub-chondrally, intra-synovially, or intrachondrally and bilaterally to the synovial joint. The pharmaceutical composition may be administered in any suitable dosage. Non-limiting examples of dosages are about 0.1 mg to 0.5 mg, such as 0.1 mg, 0.2 mg, 0.3 mg, 0.4 mg, and 0.5 mg of the pharmaceutical composition. The subject may receive a dosage of about 0.5 mg to about 1.0 mg of the pharmaceutical composition, for example the subject may receive a dosage of about 1.0 mg of the pharmaceutical composition.

### Example 3: Treating a Cartilage Disorder in Human Subjects Using a Non-Viral Particle

According to the methods disclosed herein, a subject, such as a human subject, can be administered a pharmaceutical composition (e.g., an expression vector including a DNA minicircle and a non-viral particle (e.g., a lipid nanoparticle) including a nucleic acid encoding a suicide gene and a FGF-18 polypeptide or a functional fragment thereof and a pharmaceutically acceptable carrier, diluent, or excipient) to treat a cartilage disorder in the subject. To this end, the patient is administered a pharmaceutical composition (e.g., an expression vector including a DNA minicircle and a non-viral particle (e.g., a lipid nanoparticle) including a nucleic acid encoding a suicide gene and a FGF-18 polypeptide or a functional fragment thereof and a pharmaceutically acceptable carrier, diluent, or excipient). The pharmaceutical composition thereof is administered to the subject intravenously, intra-articularly, sub-chondrally, intra-synovially, or intrachondrally. The pharmaceutical composition thereof may be administered intravenously, intra-articularly, sub-chondrally, intra-synovially, or intrachondrally and bilaterally to the synovial joint. The pharmaceutical composition may be administered in any suitable dosage. Non-limiting examples of dosages are about 0.001 mg/kg to 1.0 mg/kg, such as 0.001 mg/kg, 0.002 mg/kg, 0.003 mg/kg, 0.004 mg/kg, 0,005 mg/kg, 0.015 mg/kg, 0.055 mg/kg, 0.155 mg/kg, 0.500 mg/kg, and 1.0 mg/kg of the pharmaceutical composition. The subject may receive a dosage of about 0.0011/kg mg to about 1.0 mg/kg of the pharmaceutical composition, for example the subject may receive a dosage of about 1 mg/kg of the pharmaceutical composition.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A recombinant expression vector comprising a nucleic acid encoding (1) a suicide gene, and (2) a Fibroblast Growth Factor 18 (FGF-18) polypeptide or a functional fragment thereof.

2. The expression vector of claim 1, wherein the expression vector is a viral vector.

3. The expression vector of claim 1, wherein the expression vector comprises a non-viral particle.

4. The expression vector of claim 1, wherein the expression vector comprises a hybrid viral and non-viral particle.

5. The expression vector of claim 2, wherein the viral vector is selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, a lentivirus, and a Retroviridae family virus.

6. The expression vector of claim 5, wherein the viral vector is an AAV.

7. The expression vector of claim 2, wherein the viral vector further comprises a capsid.

8. The expression vector of claim 7, wherein the capsid comprises a naturally occurring capsid or an engineered capsid.

9. The expression vector of claim 8, wherein the engineered capsid is conjugated to a ligand.

10. The expression vector of claim 8, wherein the naturally occurring capsid is an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid.

11. The expression vector of claim 6, wherein the AAV is an AAV2 or an AAV5.

12. The expression vector of claim 1, wherein the suicide gene comprises a Herpes Simplex Virus-1 Thymidine Kinase (HSV-TK) gene, a Caspase 9 (Casp9) gene, a cytosine deaminase gene, a RQR85 polypeptide, or a truncated human Epidermal Growth Factor receptor polypeptide.

13-14. (canceled)

15. The expression vector of claim 1, wherein the suicide gene encodes an amino acid sequence that has at least 85% sequence identity to the amino acid sequence of SEQ ID NO: 1.

16-20. (canceled)

21. The expression vector of claim 1, wherein the suicide gene encodes an amino acid sequence that has at least 85% sequence identity to the amino acid sequence of SEQ ID NO: 2.

22. The expression vector of claim 1, wherein the FGF-18 polypeptide encodes an amino acid sequence that has at least 85% sequence identity to the amino acid sequence of SEQ ID NO: 3.

23-26. (canceled)

27. A pharmaceutical composition comprising the expression vector of claim 1.

28. (canceled)

29. A method of treating a cartilage disorder, the method comprising administering to the subject the pharmaceutical composition of claim 27.

30-32. (canceled)

33. A method of promoting proliferation in a chondrocyte cell or a chondrocyte precursor cell, the method comprising contacting a cell with the expression vector of claim 1.

34. A method of promoting the secretion of extracellular matrix to replace cartilage, the method comprising contacting a cell with the expression vector of claim 1.

35. (canceled)

36. A kit comprising the expression vector of claim 1, and a package insert, wherein the package insert instructs a user of the kit.

37. (canceled)


SEQUENCE LISTING (25 sequences)

SEQ ID NO: 1 - DNA, 996 bp, Artificial Sequence
  Synthetic Construct
atgcctacactgctgcgggtttacatcgacggccctcacggcatgggcaagaccaccacaacacagctgctcgtggccctgggctccagagacgacattgtgtacgtgcctgagcccatgacctactggcgggtcctgggagcctctgaaaccatcgccaatatctacaccactcagcacagactggatcagggcgagatcagcgctggcgacgccgccgtggtgatgaccagcgcccagatcaccatgggcatgccctacgccgtcaccgacgccgtgctggctcctcatattggcggcgaagccggtagcagccacgcccctcctccagctctgacccttatcttcgaccggcaccccatcgccgctctgctgtgctaccccgctgctagatacctgatgggctccatgacacctcaggccgtgctggccttcgtggccctgatcccccccaccctgcctggcacaaacatcgtgctgggcgccctgccagaagatagacacatcgatagactggccaagcggcagcggccaggcgagagactggatctggccatgctggctgctatccgcagagtgtatggcctgctggccaataccgtgagatacctgcagggcggcggaagctggagagaggactggggccagctgagcgggaccgccgtgcccccccagggcgccgagcctcaaagcaacgccggccctagaccccacatcggcgatacgctgtttaccctgttcagagcccctgagctgctggcccctaacggcgacctgtacaacgtgtttgcctgggccctcgacgtgctggctaaaagactgaggcctatgcacgtgttcatcctggactacgaccagtctcctgccggatgtagagatgcactgctgcaactgacaagcggcatgatccagacccacgtgaccacacctggatctatccctaccatctgcgacctggccagaacattcgcccgggaaatgggagaggccaactaa

SEQ ID NO: 2 - DNA, 1554 bp, Unknown
  Human alphaherpesvirus 1
atggcctctagaatcctgtggcatgagatgtggcacgagggcctggaagaggctagtagactgtacttcggcgagcggaacgtgaagggcatgttcgaggtcctggaacccctgcacgccatgatggaaagaggccctcagaccctgaaggaaacaagcttcaaccaggcctatggccgcgatctgatggaagcccaggagtggtgccggaagtacatgaaaagcgggaatgtgaaggacctgctgcaagcctgggacctgtactaccacgtgttcagacggatcagcaagctggaatacagcggaggaggaagcctggaaggagttcaggtggaaaccatctctcctggcgatggcagaacattccccaaaagaggacagacctgtgtggtgcactacaccggcatgctggaagatggaaagaaattcgacagcagcagagaccggaacaagcctttcaagttcatgctgggcaaacaggaggtgatcagaggctgggaggaaggcgtggcccaaatgagcgtgggccagcgggccaaactgacaatttctcctgactacgcctacggcgctaccggacaccctggcatcatccccccccacgccaccctggtctttgatgtggagctgctgaagctggagagcggaggcggcggctctggaggcggtggcagcggcggcggcggttccggcgtggacggcttcggcgacgtgggcgccctggaaagcctcagaggcaacgccgatctggcttacattctgagcatggagccttgtggccactgcctgatcatcaacaacgtgaacttctgcagagagagcggactgcgcaccagaaccggcagcaacatcgactgcgagaagctgcggagaagattcagctctctgcacttcatggtcgaggtgaaaggagatctcaccgccaagaagatggtgctggccctgctcgaactggcccagcaggaccacggcgccctggactgctgcgtggtggtcatcctgagccatggctgtcaggctagccacctgcagttccctggagcagtgtacggcaccgacggttgccctgtgtccgtggaaaagatcgtgaacatcttcaacggcacatcctgccccagcctgggcggcaagcccaagctgttcttcatccaggcttgtggcggcgaacagaaggaccacggcttcgaggtggccagcacaagccccgaggacgagtctccaggctctaatcctgagccagacgccaccccttttcaggagggcctgagaacttttgaccagctggatgccatctccagcctgcctacacctagcgatatcttcgtgtcatatagcacctttccaggattcgtgagctggcgggaccctaagagcggctcctggtacgtggagacactggacgatatctttgagcagtgggcccacagcgaggaccttcaatctctgctgctgcgggtggctaatgccgtgtccgttaagggcatctacaagcagatgcctggctgcttcaacttcttgagaaagaagctgtttttcaagaccagcgccagctaa

SEQ ID NO: 3 - DNA, 1998 bp, Homo sapiens
gggaggcggcgatgcgcacggccggagagacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaaaccaaagtcctttttcccaaaggttctgaaaggaaaaaaaaaaaaaacaaaaaaaaagaaaaacaaagagaaagtagtactccgcccaccaacaaactccccctaactttcccaatcctctgttcctgccccaaactccaacaaaaatcgctctctggtttgcagtcatttatttattgtccgctgcaagctgccccgagacaccgcgcagggaaggcgtgcccctgggaattctccgcgcctcgacctcccgacgacagacgcctcgtccaatcatggtgaccctgccttgctcgcagttctggaggatgctgctatcgaccttccgtgactcacgtgacctagtacaccaatgataagggaatattttaaaaccagctatattatatatattatatatatataagctatttatttcacctctctgtatattgcagtttcatgaaccaagtattactgcctcaacaattaaaaacaacagacaaattatttaaaaaacca

SEQ ID NO: 4 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 5 - protein, 9 aa, Artificial Sequence
  Synthetic Construct
SWSHPQFEK

SEQ ID NO: 6 - protein, 25 aa, Mus musculus
GCKPCICTVPEVSSVFIFPPKPKDV

SEQ ID NO: 7 - protein, 20 aa, Artificial Sequence
  Synthetic Construct
ICTVPEVSSVFIFPPKPKDV

SEQ ID NO: 8 - protein, 23 aa, Mus musculus
KPCICTVPEVSSVFIFPPKPKDV

SEQ ID NO: 9 - protein, 12 aa, Cricetulus griseus
SVFIFPPKPKDV

SEQ ID NO: 10 - protein, 7 aa, Oncorhynchus mykiss
PPKPKDV

SEQ ID NO: 11 - protein, 23 aa, Artificial Sequence
  Synthetic Construct
PCICTVPEVSSVFIEFPPKPKDV

SEQ ID NO: 12 - protein, 21 aa, Artificial Sequence
  Synthetic Construct
MWWRLWWLLLLLLLLWPMVWA

SEQ ID NO: 13 - protein, 20 aa, Artificial Sequence
  Synthetic Construct
METDTLLLWVLLLWVPGSTG

SEQ ID NO: 14 - protein, 22 aa, Homo sapiens
MDMRVPAQLLGLLLLWLRGARC

SEQ ID NO: 15 - protein, 16 aa, Homo sapiens
MPLLLLLPLLWAGALA

SEQ ID NO: 16 - protein, 23 aa, Homo sapiens
MDAMKRGLCCVLLLCGAVFVSPS

SEQ ID NO: 17 - protein, 16 aa, Artificial Sequence
  Synthetic Construct
MLLLLLLLLLLALALA

SEQ ID NO: 18 - protein, 17 aa, Homo sapiens
MLLLLLLLGLRLQLSLG

SEQ ID NO: 19 - protein, 17 aa, Artificial Sequence
  Synthetic Construct
MGVKVLFALICIAVAEA

SEQ ID NO: 20 - protein, 18 aa, Homo sapiens
MKWVTFISLLFLFSSAYS

SEQ ID NO: 21 - protein, 18 aa, Homo sapiens
MAFLWLLSCWALLGTTFG

SEQ ID NO: 22 - protein, 14 aa, Homo sapiens
MQLLSCIALILALV

SEQ ID NO: 23 - protein, 15 aa, Homo sapiens
MNLLLILTFVAAAVA

SEQ ID NO: 24 - protein, 25 aa, Homo sapiens
MALWMRLLPLLALLALWGPDPAAAF

SEQ ID NO: 25 - protein, 27 aa, Homo sapiens
MYSAPSACTCLCLHFLLLCFQVQVLVA